(19) United States
(12) Patent Application Publication
(10) Pub. No.: US 2017/0112887 A1
(43) Pub. Date: Apr. 27, 2017

(54) METHOD FOR THE ATTENUATION OF ATHEROSCLEROSIS CONDITION(S) USING LICORICE EXTRACT AS A MEDICAMENT

(71) Applicants: Yacov Fogelman, Givat Ela (IL); Yiphtah Arad, Givat Ela (IL); Efraim Zisman, Haifa (IL)

(72) Inventors: Yacov Fogelman, Givat Ela (IL); Yiphtah Arad, Givat Ela (IL); Efraim Zisman, Haifa (IL)

(21) Appl. No.: 15/292,136

(22) Filed: Oct. 13, 2016

Related U.S. Application Data
(60) Provisional application No. 62/246,064, filed on Oct. 25, 2015.

(51) Int. Cl. A61K 36/484

(52) U.S. Cl. CPC A61K 36/484

(57) ABSTRACT

The present invention is a method for the attenuation treatment of atherosclerosis condition(s), as manifested by the reduction of the carotid intima-media thickness (CIMT). The attenuation treatment is based on consuming an effective amount of a medicament comprising licorice extract which is water insoluble and free from glycyrrhizinc acid on a daily basis for a period of at least one year.

### FIELD OF THE INVENTION

The present invention relates to a method for the attenuation of atherosclerosis condition(s), as manifested by the reduction of the carotid intima-media thickness (CIMT), by the daily consuming an effective amount of licorice extract as a medicament for a lengthy time period.

### BACKGROUND OF THE INVENTION

Atherosclerosis condition or hardening of the arteries, is defined by the Merriam-Webster dictionary (web site: http://www.merriam-webster.com/dictionary/atherosclerosis) as a medical condition which is “characterized by atheromatous deposits in and fibrosis of the inner layer of the arteries”. The atheromatous deposits are typically referred to as “plaques” that buildup inside the arteries. Plaques are described as “a mass of yellowish fatty and cellular material that forms in and beneath the inner lining of the arterial walls” (medical dictionary of Dictionary.com in web site: http://dictionary.reference.com/browse/atherogenic). Plaque buildup is made of cholesterol, fatty substances, cellular waste products, calcium and fibrin (a clotting material in the blood).

Plaques may partially or totally block the blood's flow through an artery in the heart, brain, pelvis, legs, arms or kidneys. The condition varies in its severity in accordance to the degree of blockage of the blood flow and the place in the patient's body where the blockage is. When atherosclerosis condition(s) progresses to become either a physiological body-functioning limiting cause in a patient or a threat to become a physiological body-functioning limiting cause in a patient, the atherosclerosis condition(s) is regarded as an atherogenic disease. Atherogenic diseases are typically categorized as a cardiovascular disease.

Typically, the state of development (if any) of the atherosclerosis condition in a patient can be assessed (quantified) by measuring the carotid intima-media thickness (CIMT) caused by plaques buildup. In a non-invasive procedure, the measuring of the CIMT can be done by utilizing an ultrasound device (Stein J H, Korcarz C E, Hurst R T, Lonn E, Kendall C B, Mohler E R, et al. Use of carotid ultrasound to identify subclinical vascular disease and evaluate cardiovascular disease risk: a consensus statement from the American Society of Echocardiography Carotid Intima-Media Thickness Task Force. Endorsed by the Society for Vascular Medicine. J Am Soc Echocardiogr. 2008; 21:93-111. doi: http://dx.doi.org/10.1016/j.echo.2007.11.011. [PubMed]

The term “attenuate” is defined by the Merriam-Webster dictionary website as: “reduced, especially in thickness, density, or force”.

An increase in CIMT measurements over a determined time span indicates a progression in the severity of the atherosclerosis condition while an attenuation indicates a reversal in the severity of the atherosclerosis condition.

The term “medical treatment” in context of the treatment of atherosclerosis conditions is often used in an ambiguous manner. It is typically used to describe three categories of treatments:
1) A treatment procedure (or procedures) for the prevention of the start of development of atherosclerosis conditions. The treatment referred to in the context of the present text as a “prevention treatment”,
2) A treatment procedure (or procedures) to prevent further progression of the severity of the of atherosclerosis conditions after the atherosclerosis conditions have been determined (diagnosed). The treatment referred to in the context of the present text as a “maintenance treatment”
3) A treatment procedure (or procedures) for the attenuation of atherosclerosis conditions after determining the presence of atherosclerosis conditions. The treatment reverses the state and reduce the thickness of the plaques in the arteries of a patient and is referred to in the context of the present text as an “attenuation treatment”.

Plaque builds up inside the arteries is caused mainly from the depositions of cholesterol. Cholesterol is transported through the bloodstream by lipoproteins. The two types of lipoproteins that carry cholesterol to and from cells are low-density lipoprotein (LDL) and high-density lipoprotein, or (HDL), respectively. LDL cholesterol is considered the “bad” cholesterol because it contributes to plaque formation. HDL cholesterol is considered “good” cholesterol because it helps remove LDL cholesterol from the arteries. HDL acts as a scavenger, carrying LDL cholesterol away from the arteries and back to the liver, where it is broken down and passed from the body. (Source of information: The American Heart Association, website:
http://www.heart.org/HEARTORG/Conditions/Cholesterol/AboutCholesterol/Good-vs-Bad-Cholesterol_UCM_305561_Article.jsp)

The consumption of the root of the plant licorice (Glycyrrhiza glabra) as sweet flavoring agent as well for medicinal use for a wide range of aliments is recognized in traditional medicine from ancient times in both Western as well as Eastern cultures. The US National Library of Medicine (in it's website:
http://www.heart.org/HEARTORG/Conditions/Cholesterol/WhyCholesterolMatters/Atherosclerosis_UCM_305564_Article.jsp) provides a list of medical conditions in which the use of licorice may be beneficial.

U.S. Pat. No. 7,572,470 (Luba Cohen), titled: Licorice extract for use as a medicament, states that by administering an effective dose of licorice extract to patients that are at a health risk of developing atherosclerosis the development of the atherosclerosis is prevented. U.S. Pat. No. 7,572,470 quotes an article by: Fuhrman et al., published in: the American Journal of Clinical Nutrition 1997:66; 267-75, titled: Licorice extract and its major polyphenol glabridin protect low-density lipoprotein against lipid peroxidation: in vitro and ex vivo studies in humans and in atherosclerotic apolipoprotein E-deficient mice. The publication discloses that a certain ethanolic extract of licorice is useful in the inhibition of LDL oxidation. The article also reports that the consumption of licorice extract by humans did not have a significant influence on the plasma cholesterol and LDL concentration.

In U.S. Pat. No. 7,572,470 the licorice extraction is done by submerging the root material in absolute ethanol (1 kg to 5 liters) at room temperature for five hours while stirring. The ethanol is evaporated and the resultant residue dried to constant mass at 85° C. to provide 30 grams of product. The product is then pulverized to give a fine reddish brown extract that is practically insoluble in water and with no traceable amount of glycyrrhizinc acid. It is noted that other solvent may by used for extracting licorice root material, such as: acetone, ethyl acetate or hexane, but ethanol was found to give the extracts with the highest yield and activity. In the text that follows the term “licorice extract” refers to the substance obtained from the licorice root material extraction described in U.S. Pat. No. 7,572,470.

In 2002 an article by Fuhrman et al. published in: Nutrition 2002, 18:268-273 with the title: Anti-atherosclerotic effects of licorice extract supplementation on hypercholesterolemic patients; increased resistance of LDL to atherogenic modifications, reduced plasma lipid levels, and decreased systolic blood pressure. The article reported that resistance of LDL to atherogenic modification, reduced plasma lipid levels and decreased systolic blood pressure. The conclusion in the article was that “the dietary consumption of licorice-root extract by hypercholesterolemic patients may act as a moderate hypercholesterolemic nutrient and potent antioxidant agent and, hence, against cardiovascular disease”. The article 2002 of Fuhrman et al. refers to the prevention of clinical symptoms that lead to cardiovascular disease, not to an attenuation treatment after the cardiovascular disease preconditions have been formed.

In the Pakistan Journal of Nutrition 6(4)313-317 (2007) an article by: Asgary S. et al. with the title: Effect of Glycyrrhiza glabra extract on aorta wall atherosclerotic lesion in hyperchloesterolemic rabbits, was published. The article discloses that providing rabbits for 60 days with ethanol extractions of licorice roots while feeding them with a high cholesterol diet significantly prevented the development of carotid intima-media plaques in comparison to a rabbit control group that was fed with only a high cholesterol diet. The publication states that the assessment of the plaques, referred to as “atherosclerotic lesions”, were measured at the end of the experiment after the rabbits were anesthetized and the animal's aortas were excised “to study the fatty streaks”.

Asgary S. et al. (2007) states in his publication that as plaques are formed in the inner lining of the arterial walls a cascade of physiological changes take place. These changes have to do with the endothelial cell layer of the arteries as well as macrophages and muscle cells. In an advanced stage, an inflammatory reaction (also) takes place. As plaques develop, the cholesterol that initiates the process becomes entangled with physiological changes in the arterial wall. After the formation of plaques, HDL can no longer readily remove the endothelial-cell-fixated cholesterol and remediate the physiological changes that took place.

The publications previously quoted describe means (methods) for preventing the initiation of the development of atherosclerosis condition(s) as well as preventing the continual progression of the atherosclerosis condition(s) after the atherosclerosis condition(s) has developed.

The present invention relates to a method of the attenuation of atherosclerosis conditions of a patient, as manifested by the decrease of the carotid intima-media thickness (CIMT), using licorice extract as a medicament.

### SUMMARY OF THE INVENTION

Various publications, including publications quoted in the present text, have given explanations how and why the exposure of the arteries to low-density lipoprotein (LDL) that carry cholesterol contributes to the formation of plaques. The consumption of practically insoluble in water licorice root biomass, as described in U.S. Pat. No. 7,572,470, effects the oxidation of the LDL and was shown to prevent the development of plaques. Thus, the consumption of licorice extract is a “preventive treatment” for atherosclerosis conditions. The deposition and accumulation of cholesterol on the walls of the arteries is a continuous process as long as the activity of the LDL carrying cholesterol is not blocked. Asgary S. et al. (2007) have shown (in experiments with rabbits) that the consumption of licorice extract after plaques have already developed will arrest the continuation of the deposition and accumulation of cholesterol on the walls of the arteries, thus, the consumption of licorice extract may be regarded also a “maintenance treatment” for atherosclerosis conditions.

The present invention presents an “attenuation treatment” of atherosclerosis conditions. The consumption of licorice extract after the formation of plaques causes an attenuation of the plaques (attenuation of the carotid intima-media thickness) so as to reverse the severity of the atherosclerosis conditions of effected patients.

The attenuation effect of plaques in atherosclerosis conditions by the consumption of licorice extract is reported in the results of a clinical experiment, initially presented in U.S. provisional patent 62/246,064. The clinical experimental results were later published in an article in: Food Nutr. Res. 2016 Apr. 22; 60:30830 by: Fogelman Y. et al., under the title: “Antiatherosclerotic effects of licorice extract supplementation on hypercholesterolemic patients: decreased CIMT, reduced plasma lipid levels, and decreased blood pressure” (article found in website: http://www.ncbi.nlm.nih.gov/pubmed/27113136).

The clinical experiment included 110 individuals with hypercholesterolemial, susceptible to atherogenic diseases. An experimental sub-group consumed 0.2 g/day of ethanolic extract of licorice root for 12 months, while the control sub-group received a placebo. CIMT was measured at start of the experiment (base line values) and after a year. Mean CIMT was 0.92±0.25 mm at baseline in the experimental group and was significantly lower at the second assessment, after one year (0.84±0.21 mm, p=0.000). In the control group, mean CIMT at baseline was 0.85±0.17 and was significantly higher at the second assessment, after one year (88±0.19).

A full description of the clinical experiment is given in the detailed-description-of-an-embodiment section of the present document.

The present invention is a method for attenuation of atherosclerosis artery conditions, as manifested by CIMT measurements, by the consumption of an effective amount of licorice extract as a medicament on a daily basis for a lengthy time period. The term “lengthy time period” refers to clinical experimental results which indicated an attenuation of atherosclerosis conditions after a full year period of licorice extract consumption. It is reasonable to assume that the attenuation effect developed gradually and did not suddenly materialize at the very end of the full year of licorice extract consumption. It is also reasonable to assume that a further attenuation effect continued after the termination of the full year of the experiment and the continual consumption of licorice extract. Thus, the time frame for the “attenuation treatment” from the time atherosclerosis condition(s) is determined (diagnosed) in a patient till a significant CIMT attenuation is achieved, is typically a year or somewhat less than a full year.

### BRIEF DESCRIPTION OF THE DRAWINGS

[In order to better understand the present invention, and appreciate its practical applications, the following Figures (photographs) and are provided and referenced hereafter.

It should be noted that the Figures (photographs) are given as examples only and in no way limit the scope of the invention.

FIG. 1 shows a typical and general screen photograph of a CIMT-ultrasound measuring device indicating the intima-media thickness (IMT).

FIG. 2 shows a typical screen photograph of a CIMT-ultrasound measuring device indicating an electronic measurement along a centimeter in the distal common carotid artery having a normal (healthy) width of 0.6 mm.

FIG. 3 shows a typical screen photograph of a CIMT-ultrasound measuring device indicating an electronic measurement along a centimeter in the distal common carotid artery having a moderately increased IMT of 1.35 mm width.

### DETAILED DESCRIPTION OF AN EMBODIMENT

The present invention is a method of the attenuation of atherosclerosis conditions by consuming an effective amount of licorice extract as a medicament on a daily basis for a for a period of, but not limited to, a full year. The terms “consumption” and “effective amount” in context of the present invention, relates to the administering of a daily amount, but not limited to, 0.2 grams licorice extract.

An embodiment of the present invention is presented by describing in detail a clinical experiment conducted by Fogelman Y., Gaitini D., and Carmeli E., of (respectively):
the Department of Family Practice, Leumit Health Services, The Ruth & Bruce Rappaport Faculty of Medicine, Technion—Israel Institute of Technology, Haifa, Israel,
the Department of Medical Imaging, Unit of UltraSound, Rambam Health Care Center, Haifa, Israel,
the Department of Physical Therapy, Faculty of Social Welfare and Health Sciences, Haifa University, Haifa, Israel.

The clinical experiment was published in an article in: Food Nutr. Res. 2016 Apr. 22; 60:30830 under the title: “Antiatherosclerotic effects of licorice extract supplementation on hypercholesterolemic patients: decreased CIMT, reduced plasma lipid levels, and decreased blood pressure” (article found in website: http://www.ncbi.nlm.nih.gov/pubmed/27113136).

For describing the embodiment of the present invention, sections of the article describing the experimental procedure, the obtained (experimental) data, the analysis of the data and the conclusions from of the experimental data are quoted below.

The aim of the clinical experiment: Carotid intima-media thickness (CIMT) is commonly used as a predictor of cardiovascular outcome in patients with metabolic conditions such as obesity, diabetes, and cardiovascular diseases (CVDs). The easy applicability and noninvasiveness of ultrasound render it suitable as a surrogate endpoint for measuring atherosclerotic burden in people with hypercholesterolemia risk factors. The aim of this study was to analyze the anti-atherogenic effect of licorice-root extract consumption in patients diagnosed with hypercholesterolemia, according to changes in LDL and CIMT.

The Design of the experiment (quoted from the article): Individuals with hypercholesterolemia (total cholesterol≧6.18 mmol/L [240 mg/dL]) and without significant stenosis were randomly allocated to two groups: an experimental group that consumed 0.2 g/day of ethanolic extract of licorice root for 12 months, and a control group that received a placebo.

See Figures (pictures) for a visual demonstration of the CIMT measurement procedure.

Outcome measures: CIMT was measured for all study participants before and one year after starting consumption of licorice or placebo. CIMT is defined as the distance between the luminal-intima and medial-adventitia interfaces at the distal 10 mm of the common carotid artery.

Ultrasound examination of the carotid arteries was performed with patients in the supine position, with the head slightly hyper extended and turned to the opposite side. Axial and sagittal scans were obtained using a high-resolution linear transducer of 5-12 or 3-9 MHz (Philips IU22, Bothell, Wash.). Wall thickness and atherosclerotic plaques in the carotid arteries were searched for. CIMT was measured on the far wall of the distal common carotid artery on each side, 1-2 cm proximal to the bifurcation. Electronic software was applied, which follows the two echogenic lines on the far wall of the carotid artery: the lumen-intima interface and the media-adventitia interface. CIMT measurements were performed according to an international consensus statement, by skilled personnel working in the UltraSound Unit, Department of Medical Imaging.

The normal (healthy) CIMT in adults is in the range of 0.6-0.8 mm. Slightly increased, moderately increased, and severely increased CIMT are in the ranges of 0.8-1.0, 1.0-1.5, and above 1.5 mm, respectively.

Figures (photographs) 1 to 3 demonstrate the CIMT measuring technology.

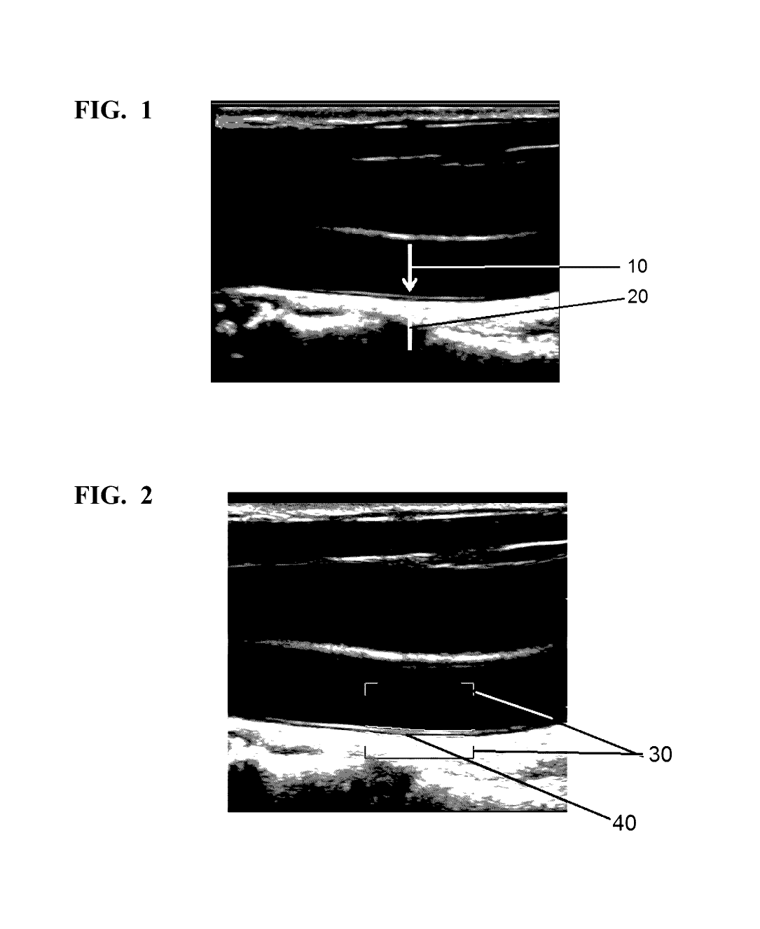

FIG. 1 presents a typical screen photograph of a CIMT-ultrasound measuring device showing the intima-media thickness (IMT). The IMT is measured between the blood-intima (upper arrow, numerated (10)) to media-adventitia (lower arrow, numerated (20)).

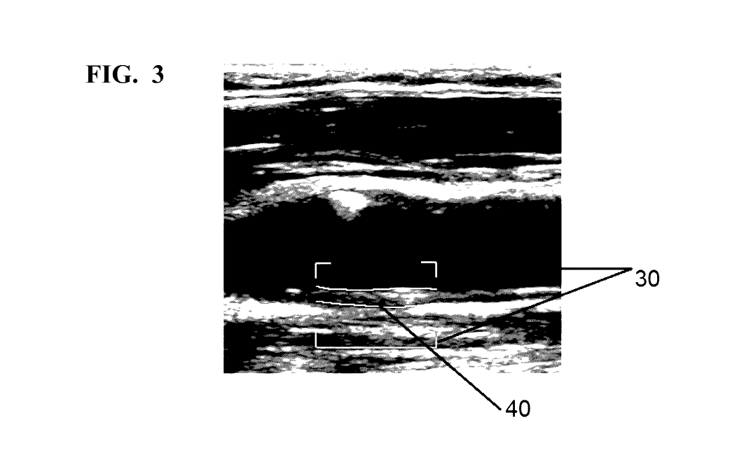

FIG. 2 and FIG. 3 present electronic measurements of the IMT, done by placing the cursors on the interfaces, along a centimeter at the distal common carotid artery. The cursors are numerated (30), the IMT numerated (40). In FIG. 2, a normal (healthy) IMT is shown, having a 0.6 mm width. In FIG. 3, a moderately increased IMT, having a 1.35 mm width, is shown.

Categorization of the outcome measures: Progression and regression in carotid atherosclerosis were defined as an increase and decrease, respectively, in the CIMT from baseline to 12 months, following daily licorice or placebo consumption. The change was measured in the same pre-selected carotid artery segments.

Statistical analysis: All analyses were carried out with the SPSS Version 21 statistical package. Quantitative results were reported as mean±SD. The data were tested by analysis of variance (ANOVA), and the means were compared across groups by Tukey's test; significance was considered as p<0.05.

The obtained results: Of 110 eligible participants, 94 (41-80 years old) completed the study. A significant CIMT decrease from 0.92±0.25 mm to 0.84±0.21 mm was observed in the experimental group compared with an increase from 0.85±0.17 mm to 0.88±0.19 mm in the control group.

Conclusion of the experiment: Following 1 full year of licorice consumption, mean CIMT (as well as total cholesterol, LDL levels, and blood pressure) decreased. This suggests that licorice may attenuate the development of atherosclerosis and of related cardiovascular diseases. It is assumed that a further decrease in the CIMT can be obtained if following the year the patients would have continued to consume the licorice extract on a daily basis.

A summary of the clinical characteristics of the patients in the experimental (n=51) and control group (n=43) is given below, in table 1.

TABLE 1

| | Experimental group | | Control group | |
|---|---|---|---|---|
| | Base line | After 1 year | Base line | After 1 year |
| BMI (kg/m²) | 28.3 (+/−) 0.7 | 28.2 (+/−) 3.12 | 28.1 (+/−) 2.8 | 27.9 (+/−) 2.9 |
| Mean CIMT (mm) | 0.92 (+/−) 0.25 | 0.84 (+/−) 0.21 | 0.85 (+/−) 0.17 | 0.88 (+/−) 0.19 |
| LDL (mg/dl) | 183 (+/−) 8.5 | 174 (+/−) 9.1 | 177.6 (+/−) 10.7 | 179.3 (+/−) 9.6 |
| HDL (mg/dl) | 39.8 (+/−) 9.2 | 39.9 (+/−) 9.4 | 38.6 (+/−) 8.9 | 38.9 (+/−) 8.1 |
| TC (mg/dl) | 284 (+/−) 32 | 262 (+/−) 25 | 291 (+/−) 35 | 289 (+/−) 31 |
| Systolic Blood | 138 (+/−) 12 | 125 (+/−) 13 | 136 (+/−) 15 | 137 (+/−) 13 |
| pressure (mm/hg) | | | | |
| Diastolic Blood | 92 (+/−) 9 | 84 (+/−) 10 | 89 (+/−) 11 | 90 (+/−) 8 |
| pressure (mm/hg | | | | |
| Current Smokers | 9 | 9 | 7 | 6 |

The discussion and conclusion of the article state: “Antiatherosclerotic effects of licorice extract supplementation on hypercholesterolemic patients: decreased CIMT, reduced plasma lipid levels, and decreased blood pressure”.

Also from Food Nutr. Res. 2016 Apr. 22; 60:30830: “Improvements in imaging technology have made possible noninvasive assessment and identification of early vascular changes using ultrasonography. B-mode ultrasound is a noninvasive, accurate modality that accurately visualizes the arterial wall thickness. CIMT is constituted by the combined thickness of the intima and media layers of the artery wall, measured at the far wall of the distal common carotid arteries. CIMT is measured by high-resolution B-mode ultrasound of extra-cranial carotid arteries and is the most widely accepted and the only non-invasive surrogate marker for early atherosclerotic disease. Increased CIMT is an intermediate stage in the continuum of atherosclerosis, which significantly correlates with coronary and cerebrovascular disease. Epidemiological studies have consistently reported a predictive value of increased CIMT for myocardial infarction and stroke, independent of traditional CV factors. This has been confirmed in a meta-analysis of 12 relevant general population-based studies. The current study showed a noteworthy decrease in CIMT in the experimental group compared with the control group”.

It should be clear that the description of the embodiment and attached Figures (photographs) set forth in this specification serves only for a better understanding of the invention, without limiting its scope.

It should also be clear that a person skilled in the art, after reading the present specification could make adjustments or amendments to the attached Figures and above described embodiment that would still be covered by the present invention.

We claim:

1) A method for attenuation treatment of atherosclerosis condition(s) by determining the presence of atherosclerosis condition(s) in a patient and administering to said patient an effective amount of a medicament comprising licorice extract which is water insoluble and free from glycyrrhizinc acid.

2) A method for attenuation treatment of atherosclerosis condition(s) according to claim 1, wherein determining the presence of atherosclerosis condition(s) in a patient is done using an ultrasound Carotid Intima-Media-Thickness (CIMT) measurement device.

3) A method for attenuation treatment of atherosclerosis condition(s) according to claim 1, wherein said administering effective amount to said patient of a medicament comprising licorice extract is a daily consumption of at least 0.2 gram of licorice extract which is water insoluble and free from glycyrrhizinc acid.

4) A method for attenuation treatment of atherosclerosis condition(s) according to claim 1, wherein said administering effective amount to said patient of a medicament comprising licorice extract is a daily consumption of licorice extract which is water insoluble and free from glycyrrhizinc acid for a period of at least one full year.
